(19) United States
(12) Patent Application Publication
(10) Pub. No.: US 2002/0051783 A1
(43) Pub. Date: May 2, 2002

(54) Method for producing or enhancing a T-cell response against a target cell using a complex comprising an HLA class I molecule and an attaching means

(76) Inventor: Philip Michael Savage, Bristol (GB)

Correspondence Address: FROMMER LAWRENCE & HAUG LLP, 745 Fifth Avenue, New York, NY, 10151, US

(21) Appl. No.: 09/878,158

(22) Filed: Jun. 8, 2001

Related U.S. Application Data
(63) Continuation-in-part of application No. 09/724,985, filed on Nov. 28, 2000.
Application No. 09/724,985 is a continuation-in-part of application No. PCT/GB99/01764, filed on Jun. 4, 1999.

(30) Foreign Application Priority Data
Jun. 5, 1998 (GB) 9812227.8
Apr. 12, 1999 (GB) 9908333.9

(51) Int. Cl. A61K 39/395; C07K 16/28

(52) U.S. Cl. 424/144.1; 530/388.1; 530/389.1

(57) ABSTRACT

A complex including an HLA class I molecule and attaching means for selectively attaching the HLA class I molecule to a target is disclosed, and a method is provided for producing or enhancing an immunological response against a target cell, by attaching said complex to the target cell. Where the target cell is diseased, foreign, or malignant cell, this method may be used to promote lysis of the target cell by T cells in the immune system. Where the target cell is an antigen presenting cell, this method may be used to promote proliferation of specific T cell clones. Uses include prevention and treatment of diseases including cancer, leukaemia, infectious diseases, viral infections, such as HIV, bacterial infections, such as tuberculosis, and parasitic infections such as malaria.

### RELATED APPLICATIONS

This application is a continuation-in-part of U.S. application Ser. No. 09/724,985, filed Nov. 28, 2000 as the continuation-in-part of PCT/GB99/01764, filed Jun. 4, 1999, designating the U.S. and published as WO 99/64464, with claims of priority from Great Britain application nos. 9812227.8, filed Jun. 5, 1998 and 9908333.9 filed Apr. 12, 1999. All of the foregoing applications, as well as all documents cited in the foregoing applications (“application documents”) and all documents cited or referenced in application documents are hereby incorporated herein by reference. Also, all documents cited in this application (“herein cited documents”) and all documents cited or referenced in herein cited documents are hereby incorporated herein by reference. Thus, the entire text of U.S. application Ser. No. 09/724,985 and PCT/GB99/01764 are incorporated herein by reference as if they were set out in full.

### FIELD AND SUMMARY OF THE INVENTION

In a broad aspect, the invention relates to the targeting of HLA class I peptide complexes via a monoclonal antibody delivery system to tumour cells. This has the effect of redirecting pre-existing T cell specificities, for example T cells with viral specificity, to kill tumour cells targeted with the HLA class I/peptide complexes.

In another broad aspect, the invention relates to targeting of HLA class I peptide complexes via a monoclonal antibody delivery system to an antigen presenting cell. This produces an immune response, eg. to activate and expand a CTL response to make sufficient cells to allow them to have an action against distant cells expressing this same combination of HLA class I molecule and peptide. In one embodied, the invention relates to ‘immunising’ with combination(s) of a HLA class I molecule plus peptide that could be tumour related such as Mart 1, Mage or other suitable molecule(s).

### BRIEF DESCRIPTION OF THE DRAWINGS

FIG. 10. Shows 5 graphs. In more detail, FIG. 10 shows a FACs analysis of the time course of binding of HLA-A2/M1 peptide complexes to HLA class I-ve cells (Daudi) via an antibody bridge. (Detected with an FITC conjugated anti-MHC monoclonal antibody (W6/32) (this antibody recognises HLA class I that is conformationally correct) (Ancell Ltd)

FIG. 11. Shows a bar chart, four scatterplots, a further barchart, four further scatterplots, and six further scatterplots. In more detail, FIG. 11 illustrates the result of a Tetramer FACs analysis of the cells cultured from peripheral blood cells incubated with or without the anti-CD20 B9E9-streptavidin fusion protein and HLA-A2/M1 peptide complex. The cells are dual stained with a FITC conjugated monoclonal antibody to CD8 and a PE conjugated HLA-A2 tetramer with specificity for HLA-A2/M1. FIG. 11 further shows the results of Tetramer FACs analysis of the cells cultured from PBLs incubated with either the anti-CD20 B9E9-streptavidin fusion protein alone (sample C) or the anti-CD20 B9E9-streptavidin fusion protein plus either the HLA-A2/MI peptide complex (sample F) or the HLA-A2/BMLF1 peptide complex (sample I). Cells from these samples were then dual stained with a FITC conjugated monoclonal antibody to CD8 and a PE conjugated HLA-A2 tetramer with specificity for HLA-A2/MI or HLA-A2/BMLF1 as indicated.

### DETAILED DESCRIPTION AND EXAMPLES

As is explained herein and in predecessor applications, U.S. application Ser. No. 09/724,985, filed Nov. 28, 2000, PCT/GB99/01764, filed Jun. 4, 1999, designating the U.S. and published as WO 99/64464, and Great Britain application nos. 9812227.8, filed Jun. 5, 1998 and 9908333.9 filed Apr. 12, 1999, incorporated herein by reference, antibody targeted HLA class I/peptide complexes have clear clinical value. The teachings and disclosure in the predecessor applications pertain to the present invention, and thus, attention is respectfully directed to the text of those applications, as the entire text of the predecessor applications are incorporated herein by reference as if they were set out in full. Accordingly, this text is to be read in conjunction with the text of the predecessor applications (and thus, the Examples and FIGS. herein begin their numbering from the numbering in U.S. Ser. No. 09/724,985).

### Pharmaceutical Compositions

The present invention also provides a pharmaceutical composition comprising a therapeutically effective amount of the complex(es) of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as- or in addition to- the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the composition is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or coloring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the complex(es) of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta-and gamma-cyclodextrins are most commonly used and suitable examples are described in WO-A-91/11172, WO-A-94/02518 and WO-A-98/55148.

The complex(es) of the present invention may be prepared in situ in the subject being treated. In this respect, nucleotide sequences encoding said complex(es) or parts thereof may be delivered by use of non-viral techniques (e.g. by use of liposomes) and/or viral techniques (e.g. by use of retroviral vectors) such that the said complex(es) are expressed from said nucleotide sequence(s).

In a preferred embodiment, the pharmaceutical of the present invention is administered topically.

Hence, preferably the pharmaceutical is in a form that is suitable for topical delivery.

### Administration

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components of the present invention may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the components can be administered (e.g. orally or topically) in the form of tablets, capsules, ovules, elixirs, solutions or suspensions, which may contain flavouring or coloring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (BPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the complex(es) may be combined with various sweetening or flavouring agents, coloring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

In a preferred aspect, the pharmaceutical composition is delivered topically.

It is to be understood that not all of the components of the pharmaceutical need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrastemally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the complex(es) and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

### Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending upon the need, the complex(es) may be administered at a dose of from 0.001 to 30 mg/kg body weight, such as from 0.1 to 10 mg/kg, more preferably from 0.1 to 1 mg/kg body weight.

### Formulation

The component(s) of the present invention may be formulated into a pharmaceutical composition, such as by mixing with one or more of a suitable carrier, diluent or excipient, by using techniques that are known in the art.

### pharmaceutically Active Salt

The complex(es) of the present invention may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

### Treatment

It is to be appreciated that all references herein to treatment include one or more of curative, palliative and prophylactic treatment. Preferably, the term treatment includes at least curative treatment and/or prophylactic treatment.

The treatment may be of one or more of cancer, tumour or related complaint. Treatment may be for producing/enhancing/augmenting immune response(s) in malignant illnesses such as cancer/leukaemia/lymphoma. Furthermore, treatment may be for infectious diseases including HIV and leprosy.

### Therapy

The complex(es) of the present invention may be used as therapeutic agents-i.e. in therapy applications.

As with the term “treatment”, the term “therapy” includes curative effects, alleviation effects, and prophylactic effects.

The therapy may be on humans or animals.

### EXAMPLES

The invention shall be further described in the following examples.

### Example 3

Inmunization/vaccination of a subject using HLA classI/peptide complex(es) to produce and/or amplify immune response(s) directed at particular cell surface molecule(s) may be accomplished according to the present invention. The immune response(s) thus produced are preferably directed at tumour cells which comprise the particular cell surface molecule(s) to which the immune response is produced. The form of this response is influenced by the particular HLA class I/peptide complex(es) used in the immunisation procedure(s).

In this example, use of antibody targeted HLA class I/peptide complexes to amplify a specific CTL response is demonstrated. Thus, example 3 also encompasses a method for delivering HLA class I/peptide complexes to the surface of antigen presenting cells.

In brief, it is demonstrated that biotinylated HLA-A2/peptide complexes immobilised on the surface of an antigen presenting cell via an antibody bridge cause the activation, amplification and expansion of cytotoxic T cells (CD8+ve) reactive with this specific HLA class I/peptide combination.

### FIGS. for Example 3

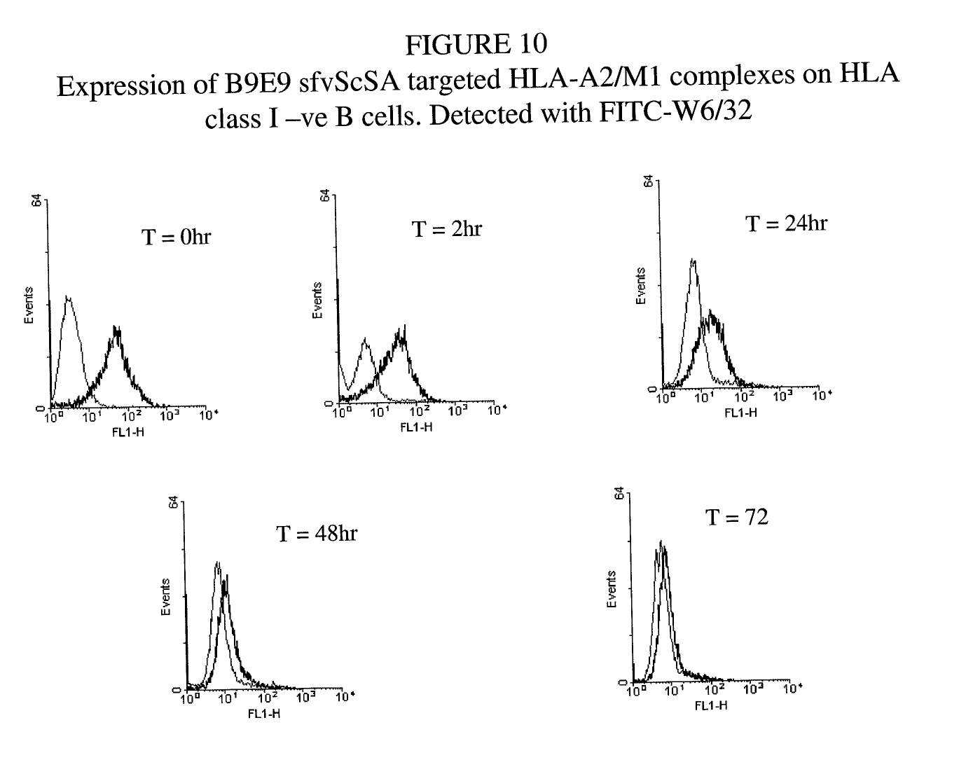

FIG. 10. Shows 5 graphs. In more detail, FIG. 10 shows a FACs analysis of the time course of binding of HLA-A2/M1 peptide complexes to HLA class I-ve cells (Daudi) via an antibody bridge. (Detected with an FITC conjugated anti-MHC monoclonal antibody (W6/32) (this antibody recognises HLA class I that is conformationally correct) (Ancell Ltd)

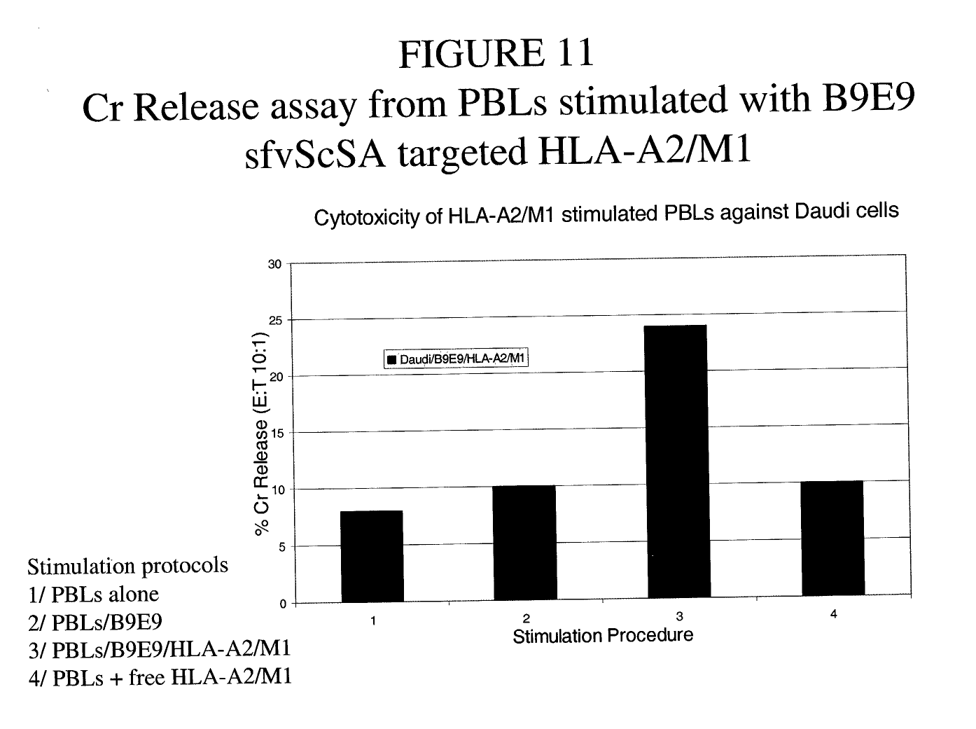

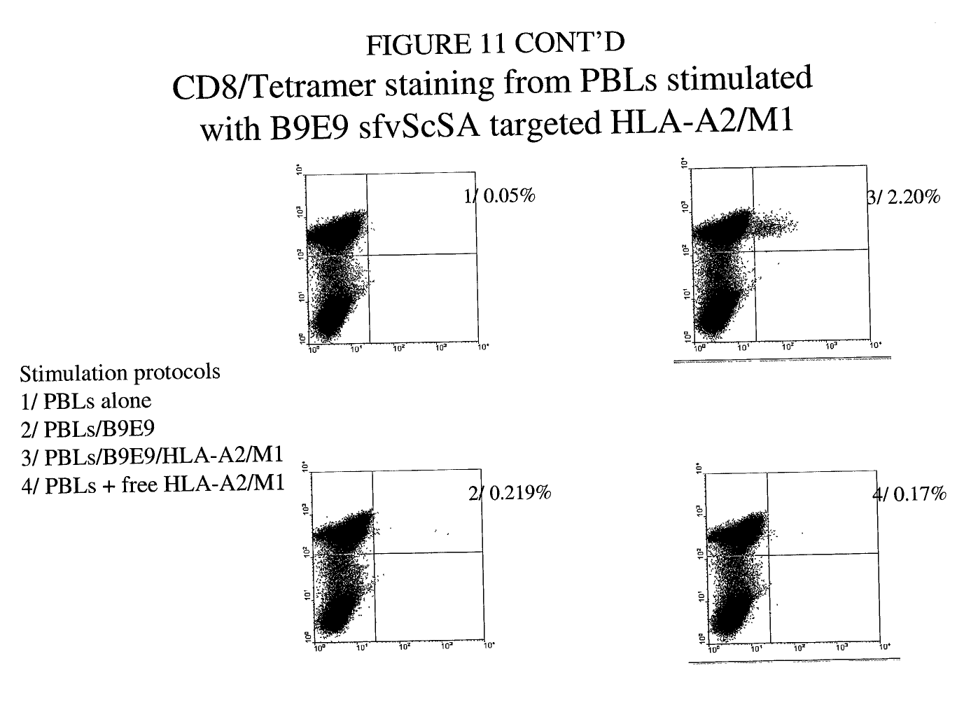

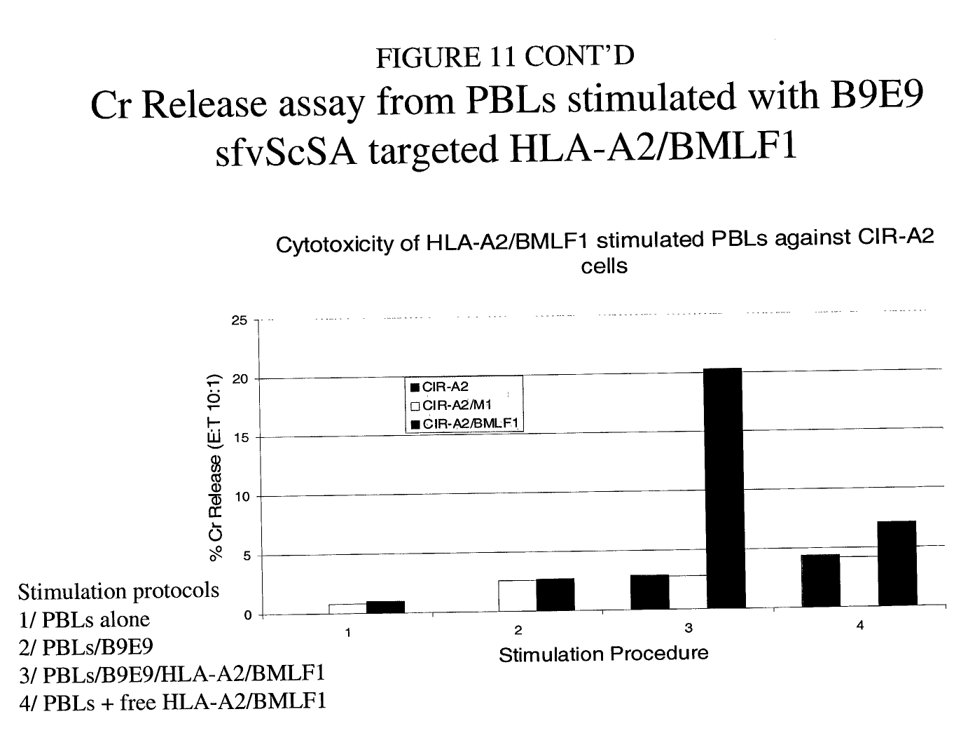

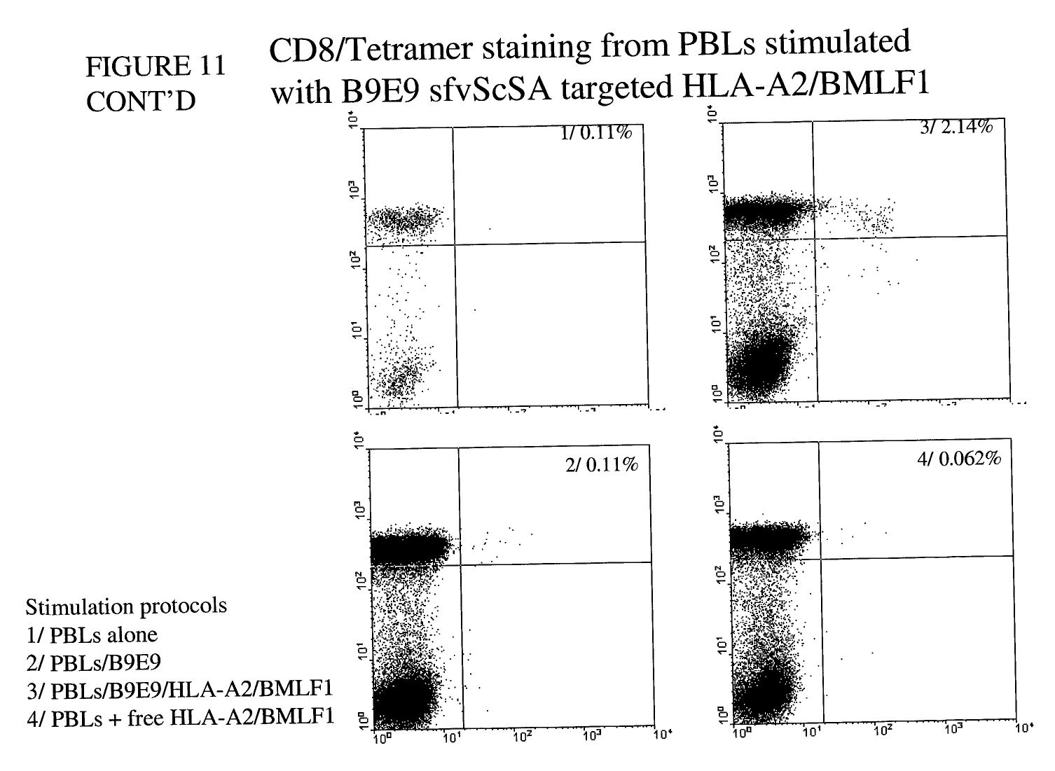

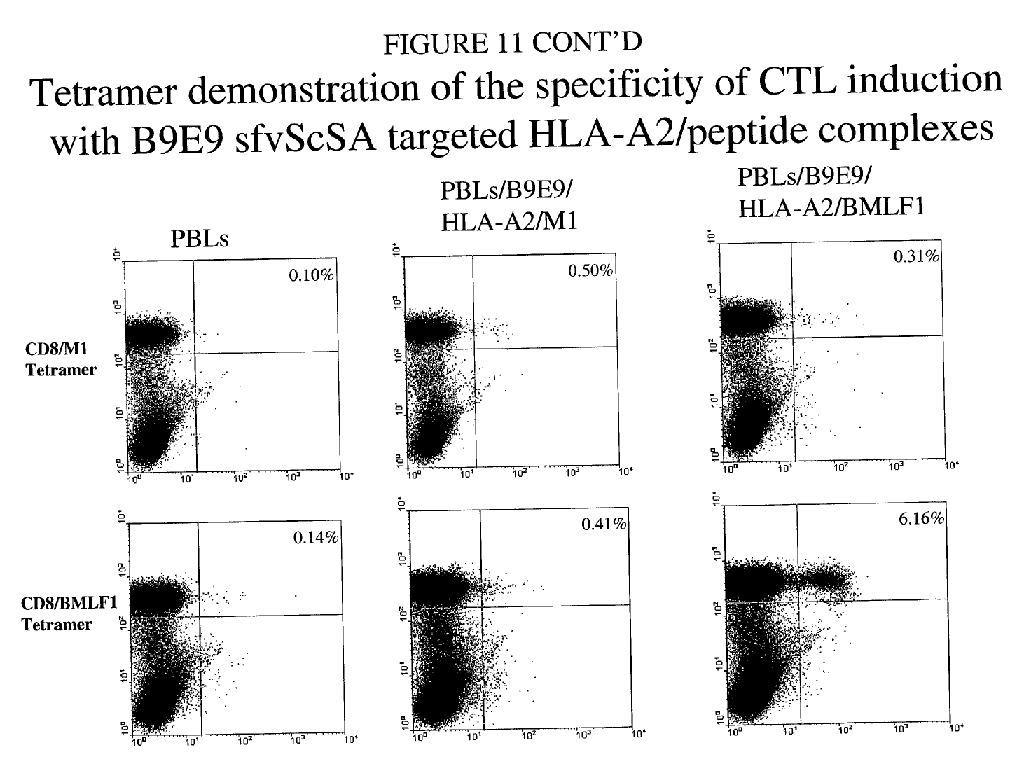

FIG. 11. Shows a bar chart, four scatterplots, a further barchart, four further scatterplots, and six further scatterplots. In more detail, FIG. 11 illustrates the result of a Tetramer FACs analysis of the cells cultured from peripheral blood cells incubated with or without the anti-CD20 B9E9-streptavidin fusion protein and HLA-A2/M1 peptide complex. The cells are dual stained with a FITC conjugated monoclonal antibody to CD8 and a PE conjugated HLA-A2 tetramer with specificity for HLA-A2/M1. FIG. 11 further shows the results of Tetramer FACs analysis of the cells cultured from PBLs incubated with either the anti-CD20 B9E9-streptavidin fusion protein alone (sample C) or the anti-CD20 B9E9-streptavidin fusion protein plus either the HLA-A2/M1 peptide complex (sample F) or the HLA-A2/BMLF1 peptide complex (sample I). Cells from these samples were then dual stained with a FITC conjugated monoclonal antibody to CD8 and a PE conjugated HLA-A2 tetramer with specificity for HLA-A2/MI or HLA-A2/BMLF1 as indicated.

Table 1(EX3): Shows the results of a T cell chromium release assay using HLA class I negative Daudi B cell lymphoma cells as targets. These cells were coated with HLA-A2/M1 peptide complexes attached via the anti-CD20 B9E9-streptavidin fusion protein.

Table 2(EX3): Shows the results of a T cell chromium release assay using CIR-A2 target cells that had been ‘pulsed’ with either nothing or the flu M1 or EBV BMLF1 peptides as indicated. The effector cells were cultured from peripheral blood cells, of an HLA-A2+ve healthy donor, incubated with the anti-CD20 B9E9-streptavidin fusion protein alone and HLA-A2/M1 or HLA-A2/BMLF1 complexes.

### Materials and Methods for Example 3

### The Following Components Were Used

Cells: Daudi cells. A human B cell lymphoma line derived from a patient with Burkitts lymphoma. The cell line was grown in RPMI standard tissue culture media.

Peripheral Blood Cells: Peripheral blood mononuclear cells from a donor previously demonstrated to be HLA-A2. These cells were separated from whole blood by differential centrifugation using Ficoll Hypaque and cultured in standard RPMI tissue culture media.

C1R-A2 cells: An HLA class I negative, human B cell line transfected with the gene for HLA-A2. These cells were cultured in RPMI tissue culture media supplemented with G418 400 ug/ml.

Attaching means: B9E9-streptavidin fusion protein, a tetrameric recombinant monoclonal antibody that binds to the B cell antigen CD20 (Schultz et al Cancer Research 2000).

HLA class I/peptide complexes: Biotin conjugated recombinant HLA class I allotype HLA-A2 molecules as described by (Altman 1996 (Science 274, 94-96)) These complexes can contain the choice of immunogenic peptides including the influenza matrix MI or EBV BMLF1 peptides. The complexes were obtained from ProImmune Ltd Oxford England. Experimental Procedures:FACs Analysis: Methods: 1/ Confirmation of ability to target HLA-A2/M1 peptide complexes to surface of B cells.

To confirm the ability of this system to target HLA-A2/M1 peptide complexes to the surface of B cells that can act as APCs, the HLA class I deficient Daudi B cell lymphoma line was used.

Approximately 1 million Daudi cells were first incubated with B9E9-SA diluted to 10 ug/ml in PBS for 1 hr at room temperature.

Following this the cells were washed twice in PBS and then incubated with biotinylated HLA-A2/M1 diluted to 1 ng/ml in PBS for 1 hr at room temperature.

The cells were then washed and placed back in a small tissue culture flask containing 5 mls of RPMI and incubated at 37° C. in a 5% CO₂ atmosphere.

The binding and time course of the residence of recombinant HLA-A2 on the cell surface of the targeted Daudi cells was demonstrated by FACs analysis.

At various time points treated Daudi cells were removed, washed in PBS and incubated for 30 minutes at room temperature with an FITC labelled anti-HLA class I monoclonal (Ancell Ltd) antibody diluted to 10 ug/ml in PBS.

After washing in PBS the cells were analysed on a Becton Dickinson FACscan machine. 2/ In vitro immunisation procedure.

30mls of whole blood was obtained from a healthy volunteer (previously documented by tissue typing to be HLA-A2+ve). Peripheral blood mononuclear cells were isolated by centrifugation using Ficoll-Hypaque.

Cells were then washed in PBS and then incubated with the B9E9 anti-CD20 streptavidin fusion protein at 10 ug/ml for 1 hr at room temperature.

The cells were then washed in PBS×2, and then incubated with the HLA-A2/peptide combination of choice at a concentration of 0.5 ug/ml for 1 hr at room temperature.

After a further wash in PBS the cells were placed in to 24 well plates at 2×10⁶ cells per well cultured in RPMI+10% FCS (heat inactivated)

On day 1 IL-7 was added to a concentration of 20 ng/ml. On day 4 and every subsequent 3 days IL-2 was added to a concentration of 10 U/ml.

The cells were incubated in a 37° C. incubator with 5% CO₂ for the duration of the experiment.

Various controls were also set up using this method including;

PBMCs alone,

PBMCs with the B9E9 antibody,

PBMCs without the B9E9 antibody but with free HLA-A2/peptide complex.

3/ Measurement of the induction/amplification of CTL activity.

The effect of the in vitro immunisation procedure on inducing the expansion of specific cytotoxic lymphocytes was assessed by two different modalities. The functional chromium release assay and the more recently described use of fluorogenic HLA class I tetramers that specifically stain cells with the desired T cell receptor specificity.

a/ Fluorogenic HLA class I Tetramer assay.

Samples from these same cells were analysed by tetramer assay.

In brief, 3×10⁵ cells were washed in PBS and then re-suspended in 100 uL of PBS. 1 uL of an HLA-A2/peptide tetramer (ProImmune, Oxford England) was added per sample and incubated for 1 hr at room temperature.

The cells were then washed in PBS and then incubated at room temperature for 1 hr with a FITC conjugated monoclonal antibody to CD8. (Dako)

After further washing in PBS the cells were fixed in a 1% solution of formaldehyde in PBS and analysed on a Becton Dickinson FACscan machine.

The results are shown in FIG. 11.

b/ The ⁵¹Cr release assay.

This assay follows standard laboratory methods. In brief CIR-A2 cells were labelled with radioactive Chromium (2 uCi/ml) by incubation for 1 hr at 37° C. in a solution of ⁵¹Cr (Amersham)

The cells were then washed and then ‘pulsed’ with a choice of immunogenic peptides at a concentration of 20 uM or no peptide at all for the negative controls.

Alternatively Daudi lymphoma cells were used as target cells. Daudi cells were coated with HLA-A2/M1 monomers by first incubating with the B9E9-streptavidin fusion protein (10 ug/ml) for 1 hr at room temperature. After washing in PBS biotinylated HLA-A2/M1 peptide complexes were added at 1 ng/ml and then incubated for 1 hr at room temperature. Following further washing the cells were used as targets in the CTL assay.

These cells were then added to round bottom 96 well plates and various numbers of the cells produced form the ‘in vitro immunization’ procedure added to give the required ‘effector:target’ ratios.

After incubation for 4 hours at 37° C., 50 uL of the supernatant was removed and the amount of ⁵¹Cr released from lysed CIR-A2 or Daudi cells was estimated using a scintillation counter.

The results are shown in Tables 1(EX3) and 2(EX3).

### Results and Discussion

1/ Demonstration of the binding and prolonged residence of HLA-A2/M1 peptide complexes targeted to CD20+ve cells via the B9E9 fusion protein.

FIG. 10. Demonstrates the time course for the retention of the HLA-A2/M1 peptide complexes bound via the B9E9/SA fusion protein to Daudi B cells.

The results demonstrate that the binding of the HLA class I/peptide complexes via the B9E9 fusion protein results in their immobilisation on the surface of these cells.

The sequential FACs analyses demonstrate an increase in signal resulting from the bound HLA class I/peptide complexes at time 0 hrs compared to the native cells. This increase over background reduces with time but is still positive after 72 hrs incubation. 2/ Demonstration by Tetramer analysis of the expansion of specific CTLs via the binding of HLA-A2/peptide complexes to B cells via CD20

### FIG. 11

### Introduction

The results of the tetramer analysis are shown both in graphical (scatterplot) and numerical form. The value of the X axis varies with the degree of tetramer bind whilst the Y axis varies with the degree of binding of the monoclonal antibody to CD8. Each dot represents an individual cell that has both an X and Y value.

The cursors are set to produce cut-off values resulting in the formation of 4 quadrants. The cells in the bottom left quadrant are judged as being negative for CD8 and tetramer binding. The cells in the left upper quadrant are positive for CD8 but negative for tetramer. The cells in the lower right quadrant are positive for tetramer but negative for CD8. The cells in the upper right quadrant are positive both for CD8 and tetramer staining, these cells are the cytotoxic T cells (CTLs) the specificity as defined by the tetramer.

### 1/ Targeting of HLA Complexes

This experiment was performed using the HLA-A2/M1 peptide complex molecule in monomeric form for the in vitro immunization and in tetrameric form for the tetramer analysis.

### RESULTS

Sample A1

These are the results from PBMCs incubated without the B9E9 fusion protein or any biotinylated HLA-A2/peptide complexes. Here only 0.089% of all the CD8+ve cytotoxic lymphocytes in the sample have specificity for the HLA-A2/M1 tetramer.

Sample A2

These are the results from PBMCs incubated with the B9E9 fusion protein but without the addition of any biotinylated HLA-A2/M1 complexes. Here 0.0287% of all the CD8+ve cytotoxic lymphocytes in the sample have specificity for the HLA-A2/M1 tetramer.

Sample A3

These are the results from PBMCs incubated with the B9E9 fusion protein and with the addition of the biotinylated HLA-A2/M1 complexes.

Here 2.19% of all the CD8+ve cytotoxic lymphocytes in the sample have specificity for the HLA-A2/M1 tetramer.

Sample A4

These are the results from PBMCs incubated without the B9E9 fusion protein but with the addition of the biotinylated HLA-A2/M1 complexes.

Here only 0.197% of all the CD8+ve cytotoxic lymphocytes in the sample have specificity for the HLA-A2/M 1 tetramer.

Further exemplary results may be found in FIG. 11.

These results show that by immobilising HLA class I/peptide complexes on to the surface of an antigen presenting cell (in this example a B lymphocyte, via a streptavin conjugated monoclonal antibody fusion protein with specificity for CD20) that a specific cytotoxic T cell response can be induced as detected by tetramer analysis.

This effect is seen when the complexes are immobilised on the cell surface-PBMCs incubated in an identical way (with IL-7 and I1-2) produce no effect (A1), neither does binding of the B9E9 fusion protein alone (A2) or the addition of free HLA class I peptide complexes (A4).

A further experiment was performed that looked at the specificity of the response to the ‘in vitro immunization’ procedure using two different HLA class I/peptide combinations and their respective tetramers.

### 2/ In Vitro Immunisation Experiment

Similar in vitro immunizations of PBMCs were set up using the following combinations of the B9E9 fusion protein and HLA-A2/M1 and HLA-A2/BMLF1 complexes. C/PBMCs with B9E9 but without any HLA-A2/peptide complex F/PBMCs with B9E9 and with the HLA-A2/M1 complex I/PBMCs with B9E9 and with the HLA-A2/BMLF1 complex

After 10 days incubation tetramer analysis using the anibody to CD8 and the PE conjugated tetramers HLA-A2/M1 and HLA-A2/BMLF1 were performed.

### Results

The results of the dual staining were;

| | CD8 + ve and | CD8 + ve and |
|---|---|---|
| | M1 tetramer + ve | BMLF1 tetramer + ve |
| Sample C | 0.021% | 0.048% |
| Sample F | 0.254% | 0.074% |
| Sample I | 0.095% | 3.80% |

These results show that the immune response from CTLs as measured by tetramer analysis is specific to the identity of the HLA class I/peptide complex used in the ‘in vitro immunization’.

In sample C which had no HLA class/peptide complex added the level of cells staining positive for CD8 and tetramer is low 0.021% for the HLA-A2/MI tetramer and 0.048% for the HLA-A2/BMLF1 tetramer.

Sample F had the HLA-A2/MI peptide complex immobilized on the B cells via B9E9 during the ‘in vitro immunization’ and here the level of HLA-A2/MI tetramer positive cells has increased over 10 fold to 0.254% whilst the HLA-A2/BMLF1 tetramer posive cells are similar to sample C at 0.074%.

The ability of the HLA-A2/BMLF1 peptide complex when immobilised on the B cells via the B9E9 fusion protein to specifically expand CTLs reactive with this peptide is shown in the results of Sample I. Here the numbers of CD8+ve cells reactive with the HLA-A2/M1 tetramer is 0.095%, which is similar to the unstimulated sample C, however now 3.80% of the CD8+ve cells bind the HLA-A2/BMLF1 tetramer, an approximately 80 fold increase in relative number.

Further exemplary results may be found in FIG. 11.

These results support the dislcosure of Experiment 1 (targeting experiment; see also predecessor applications) that the immobilised HLA class I/peptide complexes can induce a CTL response and further demonstrate that the response is specific for the HLA-class I/peptide combination. The immobilised HLA-A2/MI complex produces an expansion in CTLs that bind the HLA-A2/MI tetramer, whilst the immobilised HLA-A2/BMLF1 complex produces an expansion in CTLs that bind the HLA-A2/BMLF 1 tetramer. There appears to be little non-specific activation of CTLs of the other specificity although some modest expansion may be expected due to the release of cytokines within the cell culture. 3/ Demonstration by cytotoxicity ⁵¹Chromium release analysis of the expansion of specific CTLs via the binding of HLA-A2/peptide complexes to B cells via CD20 according to the present invention.

The chromium release assay is another method for reading out T cell activity and can give information on the functional capability of CTLs. Target cells (in this case CIR-A2 or Daudi) cells are labelled with radioactive ⁵¹Chromium and then incubated with varying numbers of ‘effector’ cells that have been produced by the ‘in vitro immunization’ procedure with the PBMCs described above.

After incubation (usually 4 hours) a sample of the cell supernatant is taken and assayed for the presence of radioactive ⁵¹ Cr which has been released from the target cells as a result of the action of specific cytotoxic lymphocytes in the effector cell population.

The Daudi cells do not express any HLA class I molecules on their cell surface. However if HLA class I/peptide complexes are attached to their surface via a monoclonal antibody they can serve as effective targets for CTLs (Ogg et al 2000).

The CIR-A2 cells serve as targets, they only possess one HLA class I allele the A2 molecule and the exact specifity of this can be altered by placing a peptide of choice within the peptide binding grove by ‘peptide pulsing’ in vitro. After performing peptide pulsing the target cells have on their cell surface of the HLA-A2 molecules a high proportion containing the peptide of choice and so form a reliable and reproducible target for CTLs of this specificity.

### Targeted Lysis Experiment

The results are expressed in terms of the degree of lysis of the target cells during the ⁵¹Cr release assay.

This is calculated according to this equation; % lysis is calculated as:

$$100 \% \times \frac{E - M}{T - M}$$

Where,

E=experimental release

M=Media release

T=Maximal release in 5% Triton 100

The ability of the following PBMC preparations to lyse Daudi cells ‘coated’ with HLA-A2/M1 peptide complexes at an E;T ratio of 10:1 was;

Sample A1

These are the results from PBMCs incubated without the B9E9 fusion protein or any biotinylated HLA-A2/peptide complexes.

Sample A2

These are the results from PBMCs incubated with the B9E9 fusion protein but without the addition of any biotinylated HLA-A2/M1 complexes.

Sample A3

These are the results from PBMCs incubated with the B9E9 fusion protein and with the addition of the biotinylated HLA-A2/M1 complexes.

Sample A4

These are the results from PBMCs incubated without the B9E9 fusion protein but with the addition of the biotinylated HLA-A2/M1 complexes.

TABLE 1(EX3)
RESULTS:
The % Lysis of HLA-A2/M1 coated Daudi cells by PBMCs
stimulated +/− HLA-A2/M1 complexes
A1 = 8%
A2 = 10%
A3 = 24%
A4 = 10%

These results demonstrate that the treatment of PBMCs with the B9E9-streptavidin fusion protein and biotinylated HLA-A2/M1 peptide complexes in accordance with the present invention results in the amplification of the CTL response to HLA-A2/M1 as measured in this assay. PBMCs treated with both parts of the system (A3) produced 24% lysis whilst the lysis produce by untreated PBMCs (A1), or PBMCs treated with the B9E9 fusion protein alone (A2) or PBMCs treated with free HLA-A2/M1 complexes produced a maximum of only 10% lysis.

### Specific Amplification Experiment

To demonstrate that the amplification of CTL response is specific to the identity of the HLA class I/peptide combination, the experiment was repeated with two HLA-A2/peptide specificities.

The results of this show differing patterns of activity for PBMCs treated with B9E9 fusion protein and the two differing HLA-A2/peptide complexes or for those treated without any HLA-A2 peptide complexes.

In this experiment CIR-A2 cells (native or peptide pulsed) were used as target cells.

The figures given are the percent lysis of peptide pulsed target cells. (E:T ratio, 5:1)

TABLE 2(EX3)
Immunization protocol:

| | | B9E9-SA + | B9E9-SA + |
|---|---|---|---|
| | B9E9-SA + | HLA-A2/M1 | HLA-A2/BMLF1 |
| Targets | Nil (sample C) | (sample F) | (sample I) |
| CIR-A2 + | 5.3% | 11.9% | 10.7% |
| Nil | | | |
| CIR-A2 + | 4.8% | 13.6% | 10.2% |
| MI | | | |
| CIR-A2 + | 7.3% | 16.4% | 23.7% |
| BMLF1 | | | |

In this experiment it is again demonstrated that PBMCs that are just targeted with the B9E9 molecule do not produce any significant lysis of target cells either native CIR-A2 or peptide pulsed with the M1or BMLF1 peptides.

PBMCs targeted with B9E9 and HLA-A2/M1 complexes produced a weak response in this particular experiment without any clear pattern of enhanced lysis.

PBMCs targeted with B9E9 and HLA-A2/BMLF1 complexes produced CLS that had enhanced activity against CIR-A2 cells pulsed with BMLF1 (23.7%) but no increased lysis against either native CIR-A2 cells (10.7%) or CIR-A2 cells pulsed with M1 peptide (10.2%).

These results further illustrate the production of a CTL response that is predominantly against the HLA class I/peptide complex which is immobilised on the surface of the antigen presenting cell according to the present invention (in this Example, B cells via CD20 using B9E9 fusion protein).

### Summary

The data in this document demonstrate that HLA-class/peptide complexes when immobilised on the surface of an antigen presenting cell via an antibody bridge result in the amplification of the immune response to that specific HLA-class/peptide complex.

The ability to specifically produce amplification of cytotoxic T cell numbers and/or activity to a particular HLA class I/peptide combination is an advantageous feature of the present invention.

The system(s) described herein offer considerable possibilities as methods for producing/enhancing/augmenting immune response(s) in malignant illnesses such as cancer/leukaemia/lymphoma. Furthermore, these systems may find application in infectious diseases including HIV and leprosy.

### Example 4 In Vivo Cancer Cell Therapy

In certain embodiments, the invention relates to using anti-viral CTLs in therapeutic approaches to combat tumour/cancer cells.

In this example, it is demonstrated that anti-viral Cytotoxic T cells inhibit the growth of cancer cells bearing antibody targeted MHC class I/peptide complexes in SCID mice.

In the present invention, cytotoxic T cells (CTLs) of non-tumour specificity are redirected against cancer cells. It is demonstrated herein that cancer cells targeted with recombinant HLA-class I/peptide complexes via an antibody delivery system can be effectively lysed by anti-viral CTLs in vitro. Furthermore, this example demonstrates effects in vivo in a mammalian system.

This system uses the recombinant anti-CD20 B9E9 sfvScSA fusion protein to target HLA-A2/M1 complexes to CD20+ve Daudi lymphoma cells. Binding of the B9E9 sfvScSA fusion protein to Daudi cells in culture had no apparent effect on growth kinetics. Using an HLA-A2/M1 specific human T cell clone, in vitro killing of targeted Daudi cells was achieved with HLA class I concentrations as low as 5 pg/ml. A tumour protection assay using human CTL to the HLA-A2/M1 complex was performed in SCID mice. Applicant demonstrates that only 1 of 4 mice receiving Daudi cells targeted with both the B9E9 sfvScSA fusion protein and the HLA-A2/M1 complex developed tumours, whilst in the control mice with receiving CTL but native Daudi cells 4 of 4 developed tumours, as did 4 of 4 receiving targeted Daudi cells but no CTLs.

This demonstration of the in vivo activity for the combination of targeted HLA class I/peptide complexes and anti-viral T cells, demonstrates the effectiveness of the antibody HLA class I targeting system. Clearly, this system may be advantageously combined with autologous CTLs produced by vaccination or ex vivo expansion.

This example also embraces aspects of the delivery (targeting) system. This example further illustrates a useful model system.

The B cell surface antigen CD20 serves as a good target for this system as it is expressed on many B cell malignancies, remains on the cell surface for days and is not internalised on antibody binding. Monoclonal antibodies to CD20 are available and are well characterised (Hainsworth 2000). Recombinant antibody fragments have also been developed. The tetravalent B9E9 sfvScSA fusion protein (see Schultz et al 2000) is useful as a targeting system.

To demonstrate the abilities of human CTLs of anti-viral specificity to interact with tumour cells targeted with HLA-A2/peptide complexes in a physiological setting, a model was developed as explained below.

Severe combined immunodeficient (SCID) mice are capable of supporting functional human CTLs for periods, possibly requiring a degree of cytokine support (de Kroon J. et al 1997, Buchsbaum et al 1996)

The human B cell lymphoma Daudi cell line can grow as a xenograft in SCID mice without requiring further routine immunosuppression and has been used in a variety of of therapeutic systems (see Gidlof et al 1997, Ghetie et al 1996).

This example demonstrates the in vivo interaction of human anti-viral CTLs and HLA targeted Daudi cells in a tumour protection experiment. Cell lines:

Clone 25—A human cytotoxic T cell clone with specificity for HLA-A2/M1 was maintained in RPMI with 10% AB serum and antibiotics.

Daudi B cell lymphoma - A CD 20+ve human B cell lymphoma cell line that is deficient for the expression of HLA class I. Daudi cells were maintained in RPMI media supplemented with 10% FCS and antibiotics in a 37° C. incubator with 5% CO₂.

Antibodies

Anti MHC class I (W6/32) Fitc conjugated (Sigma)

B9E9 sfvscSA. A recombinant tetravalent scFV/streptavidin fusion protein with specificity for CD20 (Schultz et al, 2000)

Mice

Male SCID mice aged 6-8 weeks were maintained in sterile conditions in a suitable animal facility.

Facs analysis

Becton Dickinson FACscan machine with relevant software.

Methods

Action of B9E9 scFvSA on Daudi cell growth

Daudi cells were washed in PBS and then incubated with dilutions of the B9E9 scFvSA in PBS for 1 hour at room temperature. After two washes the cells were re-suspended in 5 mls of tissue culture media and incubated at 37° C. in a 5% CO2 atmosphere. The proliferation of the antibody treated cells and controls was assessed by sequential counts of the viable cells using Trypan blue exclusion and a haemocytometer.

### Effect of HLA Dilutions on In Vitro CTL Mediated Lysis

Standard Chromium release assays were performed using the Daudi B cell line as the target cell. Briefly, cells were labelled with 100 uCi of ⁵¹ Cr (Amersham Pharmacia) for 1 hr at 37° C. After washing in PBS, cells were incubated with B9E9 at 10 ug/ml for 1 hr at room temperature. After two further washes cells were incubated with dilutions of HLA-A2/M1 complexes in PBS for 1 hour at 4° C. After 2 further washes the cells were plated out at 3000 cells per well in U-bottomed 96 well plates. Tissue culture media, dilutions of CTLs or 5% Triton X-100 were added to a final volume of 200 uL. Plates were incubated for 4 hours at 37° C. in a 5% CO2 atmosphere and then 50 uL of supernatant collected and added to 150 uL of scintillant in a standard scintillation plate and counted. The specific lysis was calculated as;

$$\% lysis = \frac{experimental cpm - spontaneous cpm}{maximum cpm - spontaneous cpm} \times 100$$

The spontaneous release was measured from the cells incubated in media alone, the maximum release was measured from the cells incubated in Triton.

Facs analysis was performed on the cells targeted with B9E9 sfvScSA fusion protein and HLA-A2/M1 prepared as above. Samples of cells were washed in PBS and then incubated with FITC labelled anti-MHC class I (Ancell, Nottingham UK) and analysed by flow cytometry on a Becton Dickinson FACscan.

### In Vivo Tumour Protection Assay

Healthy male SCID mice aged 6-8 weeks were used for the tumour protection assay. Four mice were used in each of the 3 groups A, B and C, treated as follows;

Group A

Mice in Group A were injected IP with 3×10⁶ clone 25 cells in 0.2ml of sterile PBS on Day 1. On Day 2 1×10⁶ Daudi cells targeted sequentially, ex vivo, with B9E9 sfvFSA (10 ug/ml) and HLA-A2/M1 (0.5 ug/ml) were injected IP in 0.2 ml of sterile PBS.

Group B

Mice in group B were injected IP with 3×10⁶ clone 25 cells in 0.2 ml of sterile PBS on Day 1. On Day 2 1×10⁶ native Daudi cells were injected IP in 0.2 ml of sterile PBS.

Group C

Mice in group C were injected with 0.2 ml of sterile PBS on Day 1. On Day 2 1×10⁶ Daudi cells targeted sequentially, ex vivo, with B9E9 sfvFSA (10 ug/ml) and HLA-A2/M1 (0.5 ug/ml) were injected IP in 0.2 ml of sterile PBS.

The mice in all 3 groups received IP injections with human IL-2 (Chiron) 2,500 U in 0.1 ml PBS daily on days 1-3.

Following these procedures the mice were maintained in sterile conditions and monitored for tumour development. All mice were sacrificed at day 60 and assayed for tumour development.

### Effects of B9E9 SFVSCSA Binding on Daudi Cell Kinetics In Vitro

To investigate the possibility of any apparent effect on cell kinetics from the binding of the B9E9 sfvSA fusion protein to the Daudi lymphoma cells a simple in vitro study was performed. The results from this shown in Table 1(EX4) show the growth over 4 days of the cells treated with dilutions of B9E9 sfvScSA and the untreated control cells. The rates of proliferation for the native and antibody bound cells appear comparable with no significant effect on the growth of the Daudi lymphoma cells in culture resulting from B9E9 sfvScSA binding.

TABLE 1(EX4)
The effects of B9E9 sfvSA binding to Daudi cell growth kinetics in vitro.
(Results expressed as cells × 10⁴/ml)

| Expt 1 (B9E9) | | | | | |
|---|---|---|---|---|---|
| 19.3.01) | Day 0 | Day 1 | Day 2 | Day 3 | Day 4 |
| Daudi + nil | 20 | 27 | 33 | 51 | — |
| Daudi + 0.1 ug/ml | 20 | 34 | 33 | 54 | — |
| Daudi + 1 ug/ml | 20 | 23 | 33 | 76 | — |
| Daudi + 10 ug/ml | 20 | 26 | 27 | 76 | — |
| Expt 2 (B9E9) | | | | | |
| 9.4.01) | | | | | |
| Daudi + Nil | 20 | 22 | 29 | 36 | 71 |
| Daudi + 10 ug/ml | 20 | 11 | 17 | 32 | 70 |

### Dose Response of HLA Binding Concentration Measured By Facs Signal and CR Release Assay

To investigate the effects of varying of the concentration of biotinylated HLA class I molecules delivered to the target cells a dose response was obtained using FACs analysis and Cr release assays as shown in Table 2(EX4).

The results indicate that a positive signal could be obtained by FACs with concentrations of biotinylated HLA down to approximately 1-5 ng/ml. The functional chromium release assay show that effective lysis of target cells can occur when exposed to concentrations of biotinylated HLA class as low as 5 pg/ml, which is producing levels of HLA binding that are significantly below the level of FACs detection.

TABLE 2(EX4)
HLA-A2/M1 complex concentration/ml

| | | 10 | 1 | 0.1 | 10 | 5 | 1 | 0.5 | 0.1 | 50 | 10 | 5 | 1 |
|---|---|---|---|---|---|---|---|---|---|---|---|---|---|
| | Nil | ug | ug | ug | ng | ng | ng | ng | ng | pg | pg | pg | pg |
| FACs | 5.8 | 18.98 | 22.54 | 18.41 | 8.47 | 7.40 | 5.71 | 5.8 | 4.6 | — | — | — | — |
| (Gm) | | | | | | | | | | | | | |
| ⁵¹Cr | 2% | n/d | n/d | n/d | 42% | n/d | 51% | 50% | 42% | 34% | 22% | 18% | 1% |

(4 hour chromium release assay E:T ratio 5:1)

### In Vivo Tumour Protection Assay

The ability of anti-viral CTLs to interact with cancer cells targeted with the HLA-class I peptide complexes was assayed in a tumour protection assay in SCID mice. After inoculation of the tumour cells the animals were monitored and sacrificed at day 60 when signs of disease became apparent. After sacrifice the mice were dissected and the tumour tissue weighed.

The results of the 3 groups were;

Group A (Day 1 Clone 25, Day 2 Daudi +B9E9+HLA-A2/M1)

1/Tumour mass 2.4 g

2/No tumour

3/No tumour

4/No tumour

Group B (Day 1 Clone 25, Day 2 Daudi-native)

1/Tumour mass 5.64 g

2/Tumour mass 0.66 g

3/Tumour mass 1.54 g

4/Tumour mass 2.36 g

Group C (Day 1 PBS, Day 2 Daudi+B9E9+HLA-A2/M1)

1/Tumour mass 3.78 g

2/Tumour mass 1.50 g

3/Tumour mass 3.06 g

4/Tumour mass 3.47 g

Thus, use of the cellular immune system to selectively attack cancer cells according to the present invention has been demonstrated.

There are an increasing number of tumour associated peptides described that may serve to immunologically distinguish cancer from normal cells, and may therefore be useful in targetting aspects of the present invention. Particularly suitable are tumour specific or tumour associated cell surface antigens that can be bound by monoclonal antibodies. A number of these antibodies are now available, and could be adapted to deliver complexes of the present invention to the surface of tumour cells.

The production of large numbers of CTLs reactive with viral epitopes useful in the present invention is relatively easy in vivo as a result of infection by the relevant virus(es) and/or vaccination with said viral epitope(s). These CTLs may even be prepared/supplied ex vivo by specific antigenic stimulation.

The use of antibody targeted HLA class I/peptide complexes to redirect the lytic action of CTLs having anti-viral specificity against tumour cells according to the present invention is demonstrated. Antibody targeted HLA class I tetramers may be used, and a range of tumour cells targeted may be effectively killed by anti-viral CTLs. These aspects of the present invention are further illustrated by the in vivo data presented herein.

The applicant has advantageously reduced the number of targeting steps to 2 in this example by the use of the B9E9-sfvScSA fusion protein.

To investigate if binding of B9E9 to the Daudi cells used in the in vivo examples had any effect itself on said cells, the applicant examined the effects of binding B9E9 on cell growth. The results shown in table 1 (EX4) demonstrate that there was no significant effect on cell growth. Thus, without wishing to be bound by theory, the effects observed clearly flow from the methods of the present invention, and not from a mere effect of antibody binding.

The results shown in table 2(EX4) demonstrate that significant lysis of tumour cells occurs in vitro even at very low concentrations of the biotinylated HLA class I/M1 complex. Only at levels of 50 pg/ml or below does the degree of lysis begin to reduce slightly and activity is maintained down to 5 pg/ml. The very high affinity of biotin-streptavidin interaction (10⁻¹⁵M) means that binding takes place efficiently even at these low concentrations. In a clinical scenario, with possible difficulties of targeting access, a possible degree of antigen shedding and possibly a relatively short half-life of 3 polypeptide chain types of recombinant HLA class I molecules, this advantageous feature of the present invention (ie. the ability to produce functionally effective targeting at low HLA concentrations and/or to produce effective CTL mediated lysis with only relatively small numbers of molecules immobilised on each target cell) may be very valuable.

Function of the system in vivo is demonstrated. Of the animals pre-treated with the anti-HLA-A2/M1 CTL clone 25 and then injected with targeted Daudi cells, only 1 of the 4 developed a tumour. In contrast, the control groups (ie. either mice pre-treated with clone 25 but receiving native Daudi cells, or mice with no CTL pretreatment receiving targeted Daudi cells), all 4 of each group developed tumours.

Thus, it is demonstrated that anti-viral CTLs can effectively interact in vivo with these cells and are effectively targeted with antibody targeted HLA class I/peptide complexes according to the present invention.

The low toxicity of the targeting antibody and of the recombinant HLA class I peptide complexes demonstrates that sufficient molecules may be delivered to target cells, facilitating effective CTL activity according to the present invention.

HLA stability may advantageously be improved by the production of single chain recombinant versions.

Production of CTLs by vaccination, or the administration of CTLs expanded ex vivo such as described for the treatment of EBV associated lymphoma (Savoldo et al 2000) may be advantageously employed in the present invention.

| | | Clone 25 | Clone 25 | Clone 12 |
|---|---|---|---|---|
| HLA conc | | E:T 8:1 | E:T 10:1 | E:T 5:1 |
| 10 | ug/ml | 91% | | |
| 5 | ug/ml | 38% | | |
| 1 | ug/ml | 69% | | |
| 100 | ng/ml | 69% | | |
| 10 | ng/ml | 46% | 97% | 42% |
| 1 | ng/ml | 75% | 85% | 51% |
| 0.5 | ng/ml | — | 79% | 50% |
| 0.1 | ng/ml | 49% | 87% | 42% |
| 0.05 | ng/ml | — | — | 34% |
| 0.01 | ng/ml | 27% | 44% | 22% |
| 0.005 | ng/ml | — | 26% | 18% |
| 0.001 | ng/ml | 13% | 12.8% | 1.4% |

### References

Cormier, J. N., Panelli, M. C., Hackett, J. A., Bettinotti, M. P., Mixon, A., Wunderlich, J., Parker, L. L., Restifo, N. P., Ferrone, S. and Marincola, F. M. (1999) Natural variation of the expression of HLA and endogenous antigen modulates CTL recognition in an in vitro melanoma model. Int J Cancer 80, 781-790.

Zavazava, N. and Kronke, M. (1996) Soluble HLA class I molecules induce apoptosis in alloreactive cytotoxic T lymphocytes. Nature Medicine 2, 1005-1010.

Buchsbaum, R. J., Fabry, J. A. and Lieberman, J. (1996) EBV-specific cytotoxic T lymphocytes protect against human EBV-associated lymphoma in scid mice. hmmunology Letters 52, 145-152.

Robert, B., Guillaume, P., Luescher, I., Romero, P. and Mach J -P. (2000) Antibody-conjugated MHC class I tetramers can target tumor cells for specific lysis by T lymphocytes. Eur. J Immunol 30, 3165-3170.

De Kroon, J. F. E. M., van Bergen, C. A. M., de Paus, R. A., Kluin-Nelemans, H. C. K., Willemze, R. and Falkenberg, J. H. F. (1997) Human cytotoxic CD8+T-lymphocyte clones engraft in severe combined immunodeficient (SCID) mice but show diminished function. Journal of Immunotherapy, 20, 101-110.

Ghetie, M. A., Podar, E. M., Gordon, B. E., Pantazis, P., Uhr, J. W. and Vitetta, E. S. (1996) Combinationimmunotoxin treatment and chemotherapy in SCID mice with advanced, disseminated Daudi lymphoma. Int J Cancer 68, 93-96.

Gidlof, C., Dohlsten, M., Lando, P., Kalland, T., Sundstrom, C. and Totterman, T. H. (1997) A supernatgen-antibody fusion protein for T cell immunotherapy of human B lineage malignancies. Blood 89, 2089-97.

Van Ojik, H. H. and Valerius, T (2001) Preclinical and clinical data with bispecific antibodies recruiting myeloid effector cells for tumor therapy. Crit Rev Oncol Hematol 38, 47-61.

Nielsen, S. E., Zeuthen, J., Lund, B., Persson, B., Alenfall, J. and Hansen, H. H. (2000) Phase I study of single escalating doses of a superantigen-antibody fusion protein (PNU-214565) in patients with advanced colorectal or pancreatic carcinoma. J Immunother 23, 146-53.

Penichet, M. L. and Morrison, S. L. (2001) Antibody-cytokine fusion proteins for the therapy of cancer. J Immunol Methods 248, 91-101.

Hainsworth, J. D. (2000) Rituximab as first line systemic therapy for patients with low-grade lymphoma. Semin Oncol 27, 25-9.

Savoldo, B., Heslop, H. E and Rooney, C. M. (2000) The use of cytotoxic T cells for the prevention and treatment of Epstein-Barr virus induced lymphoma in transplant recipients. Leuk Lymphoma 39, 455-464.

What is claimed is:

1. A complex comprising an HLA class I molecule or fragment thereof, which HLA class I molecule or fragment thereof comprises a T cell binding portion, arid attaching means for selectively attaching said HLA class I molecule or fragment thereof to a target cell.

2. A complex as claimed in claim 1, wherein said attaching means comprises a linking polypeptide with high specific affinity for a target cell specific molecule on the surface of the target cell.

3. A complex as claimed in claim 2, wherein said linking polypeptide comprises ail antibody, preferably a monoclonal antibody, raised against said target cell specific molecule.

4. A complex as claimed in claim 2, wherein said linking polypeptide is adapted to be attached directly to said HLA class I molecule or fragment thereof.

5. A complex as claimed in claim 2, wherein said attaching means further comprises a coupling system for coupling said linking polypeptide to said HLA class I molecule or fragment thereof.

6. A complex as claimed in claim 5, wherein said coupling system comprises a two- or three-step chain of well-characterised paired small molecules, which chain is joined to the linking polypeptide and the HLA class I molecule so as to form a stable bridge between the two.

7. A complex as claimed in claim 6, characterised in that said chain comprises biotin and avidin/streptavidin.

8. A complex as claimed in claim 6, characterised in that said chain comprises calmodulin and calmodulin binding peptide.

9. A complex as claimed in any preceding claim, which complex comprises a recombinant protein, which recombinant protein includes a moiety comprising said HLA class I molecule or fragment thereof, and a moiety comprising said attaching means.

10. A complex as claimed in claim 1, characterised in that said HLA class I molecule or fragment thereof binds or is attached to a recognition peptide, which recognition peptide is arranged to be presented by said HLA class I molecule or fragment thereof for T cell recognition.

11. A complex as claimed in claim 1, characterised in that said target cell is a type of cell the presence of which is undesirable in a patient, such as a tumour cell or a diseased, foreign or malignant cell such as a cancer cell, a leukaemia cell, a cell infected with the HIV virus or with any other parasite, bacterium, microbe or virus, or a cell responsible for detrimental activity in autoimmune disease.

12. A complex as claimed in claim 11 appended to claim 10, wherein there is a recognition peptide that comprises a peptide which has a strong cytotoxic T cell response or which is capable of inducing a powerful immune response.

13. A complex as claimed in claim 11 wherein there is a recognition peptide comprises a viral or microbial peptide, such as an influenza virus peptide, a measles virus peptide, an Epstein-Barr virus peptide, in particular an Epstein-Barr virus peptide comprising the RAKFFQLL epitope of the lytic protein BZLF1, a Cytomegalovirus peptide, or a tetanus toxoid peptide.

14. A complex as claimed in ay of claim 11, wherein the allotype of said HLA class I molecule or fragment thereof is different front tile allotype of tile HLA class I molecules of the patient, so that all alloreactive response call additionally or alternatively be triggered against said target cell.

15. A complex as claimed in claim 1, wherein said target cell is an antigen presenting cell.

16. A complex as claimed in claim 15, wherein there is recognition peptide that comprises a tumour specific peptide, or a viral peptide, or a bacterial peptide, or a parasitic peptide, or any peptide which is exclusively or characteristically presented by HLA class I molecules on the surface of diseased or malignant cells, or virally, bacterially, parasitically or microbially infected cells, or foreign cells the presence of which is undesirable in a patient.

17. A complex as claimed in claim 1, wherein said target cell is a culture cell.

18. A complex as claimed in claim 1, wherein said target cell is a cell in the body of a patient.

19. A method for attaching the complex of claims 1 to said target cell, comprising the step of introducing to said target cell said I-ILA class I molecule or fragment thereof and said attaching means.

20. Use of the complex of claim 15 in the in vivo or ex vivo amplification of cytotoxic T cells with specificity for said recognition peptide.

21. A method for producing or enhancing an immunological response against a target cell, comprising the step of attaching the complex of claim 1 to said target cell by introducing to said target cell a HLA class 1 molecule or fragment thereof and attaching means.

22. A method for immunising a patient against a disease or condition which is characterised by the presence in the patient's body of cells displaying said recognition peptide on the surface thereof, such as a tumour, or a malignant or auto-immune disease such as cancer or leukaemia, an infectious disease such as a viral infection such as HIV infection, a bacterial or microbial infection such as tuberculosis, or a parasitic infection such as malaria; comprising the step of administering to said patient an effective amount of the complex of claim 15.

23. A pharmaceutical composition for use in immunising a patient against a disease or condition which is characterised by the presence in the body of the patient of diseased, malignant or foreign cells; such as a tumour, or a malignant or auto-immune disease such as cancer or leukaemia, or an infectious disease such as a viral infection such as HIV infection, or a bacterial or microbial infection such as tuberculosis, or a parasitic infection such as malaria; said pharmaceutical composition comprising a complex as claimed in claim 15 and an appropriate excipient or carrier.

24. Use of the complex of claim 15 in the preparation of a medicament for use in immunising a patient against a disease or condition which is characterised by the presence in the patient's body of cells displaying said recognition peptide on the surface thereof such as a tumour, or a malignant or auto-immune disease such as cancer or leukaemia, an infectious disease such as a viral infection such as HIV infection, a bacterial or microbial infection such as tuberculosis, or a parasitic infection such as malaria.

25. A method for the treatment of a disease or condition such as a tumour, or a malignant or auto-immune disease such as cancer or leukaemia, an infectious disease such as a viral infection such as HIV infection, a bacterial or microbial infection such as tuberculosis, or a parasitic infection such as malaria, comprising the step of administering to a patient in need thereof an effective amount of the complex of claim 11.

26. A pharmaceutical composition for use in the treatment of a disease or condition characterised by the presence in a patient of diseased, foreign or malignant cells; such as a tumour, or a malignant or auto-immune disease such as cancer or leukaemia, or an infectious disease such as a viral infection such as HIV infection, or a bacterial or microbial infection such as tuberculosis, or a parasitic infection such as malaria; said pharmaceutical composition comprising a complex as claimed in claim 11 and an appropriate excipient or carrier.

27. Use of the complex of claim 11 in the preparation of a medicament for the treatment of a tumour, or a malignant or auto-immune disease such as cancer or leukaemia, or an infectious disease such as a viral infection such as HIV infection, or a bacterial or microbial infection such as tuberculosis, or a parasitic infection such as malaria.

28. A pharmaceutical pack or kit comprising one or more containers containing one or more of the pharmaceutical compositions claimed in claim 23 and written instructions for the administration of said composition or compositions to a patient.
